(19) United States
(12) Patent Application Publication
(10) Pub. No.: US 2009/0204099 A1
(43) Pub. Date: Aug. 13, 2009

(54) Cervical dilating and ripening catheter system and method

(76) Inventor: Michael Feloney, Omaha, NE (US)

Correspondence Address: SUITER SWANTZ PC LLO, 14301 FNB PARKWAY, SUITE 220, OMAHA, NE, 68154, US

(21) Appl. No.: 12/322,574

(22) Filed: Feb. 4, 2009

Related U.S. Application Data
(60) Provisional application No. 61/027,137, filed on Feb. 8, 2008.

(51) Int. Cl. A61B 17/42; A61M 25/10

(52) U.S. Cl. 604/500; 604/101.01; 606/121

(57) ABSTRACT

A cervical dilating and ripening catheter system includes an elongated tube having a first end and a second end, and a first expandable portion affixed to the second end of the elongated tube. The system also includes a second expandable portion disposed between the first expandable portion and the first end of the elongated tube. The system further includes means for distributing a cervical ripening medication to the cervix.

### CROSS-REFERENCE TO RELATED APPLICATIONS

The present application claims the benefit under 35 U.S.C. §119(e) of U.S. Provisional Application Ser. No. 61/027,137, filed Feb. 8, 2008, which is incorporated herein by reference.

### TECHNICAL FIELD

The present disclosure generally relates to the field of catheters, and more particularly to a catheter that may allow for the safe dilatation and ripening of the cervix during labor.

### BACKGROUND

A successful vaginal birth is less likely when a patient's cervix is not ripened. Traditionally, a foley catheter is used to help dilate or ripen a patient's cervix by providing mechanical pressure directly on the patient's cervix. However, some issues with the foley catheter may include the foley catheter pulling back on the patient's cervix with an unknown pressure or force.

### SUMMARY

A cervical dilating and ripening catheter system includes an elongated tube having a first end and a second end, and a first expandable portion affixed to the second end of the elongated tube. The first expandable portion is configured to be placed in a uterine cavity through a cervix. The system also includes a second expandable portion disposed between the first expandable portion and the first end of the elongated tube. The second expandable portion is configured to be placed in the cervix upon the first expandable portion being pulled against the proximal aspect of the cervix. The system further includes means for distributing a cervical ripening medication to the cervix.

A method for delivering a cervical ripening medication includes placing a first expandable portion into a uterine cavity through a cervix. The first expanding portion is affixed to a second end of an elongated tube. The method also includes positioning a second expandable portion in the cervix by pulling the first expandable portion against the proximal aspect of the cervix. The second expandable portion is disposed between the first expandable portion and a first end of the elongated tube. The method further includes delivering a cervical ripening medication to the cervix.

A cervical dilating and ripening catheter system includes an elongated tube having a first end and a second end, and a first balloon affixed to the second end of the elongated tube. The first balloon is configured to be placed in a uterine cavity through a cervix. The system also includes a second balloon disposed between the first balloon and the first end of the elongated tube. The second balloon is configured to be placed in the cervix upon the first balloon being pulled against the proximal aspect of the cervix. The system further includes a lumen for distributing a cervical ripening medication to the cervix.

It is believed that the present disclosure has advantages over the previously described foley catheter. One advantage may include that the present disclosure may deliver a cervical ripening medication to assist with the ripening of the patient's cervix. Furthermore, a Leveen like pressure inflating device will allow a user to control the second expandable portion with a known pressure.

It is to be understood that both the foregoing general description and the following detailed description are exemplary and explanatory only and are not necessarily restrictive of the present disclosure. The accompanying drawings, which are incorporated in and constitute a part of the specification, illustrate subject matter of the disclosure. Together, the descriptions and the drawings serve to explain the principles of the disclosure.

### BRIEF DESCRIPTION OF THE DRAWINGS

The numerous advantages of the disclosure may be better understood by those skilled in the art by reference to the accompanying figures in which:

FIG. 1 is an exploded view of a cervical dilating and ripening catheter system;

FIG. 2 is an embodiment of the cervical dilating and ripening catheter system of FIG. 1, applied to a patient, shown in cross section;

FIG. 3 is a partial view of the cervical dilating and ripening catheter system illustrated in FIG. 1, applied to a patient;

FIG. 4 is a partial view of the cervical dilating and ripening catheter system illustrated in FIG. 1, applied to a patient, wherein a first expandable portion is pulled against a proximal aspect of a cervix; and

FIG. 5 is a flow diagram illustrating a method for delivering a cervical ripening medication.

### DETAILED DESCRIPTION

Reference will now be made in detail to the subject matter disclosed, which is illustrated in the accompanying drawings.

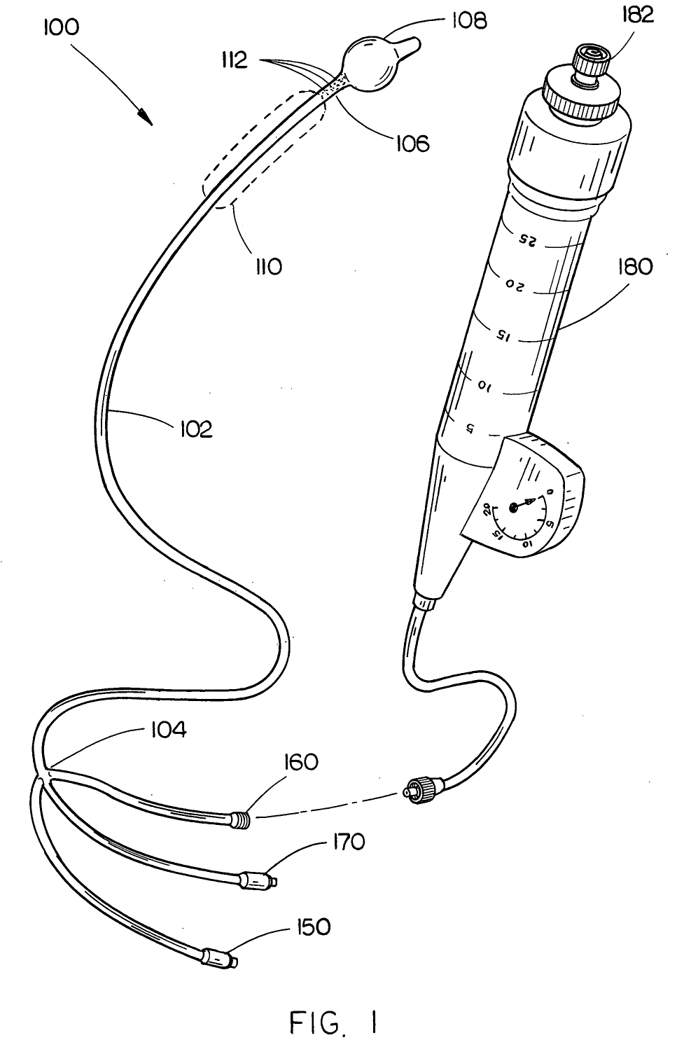

Referring to FIGS. 1-4, diagrams illustrating embodiments of a cervical dilating and ripening catheter system 100 are shown. The system 100 may include an elongated tube 102 that has a first end 104 and a second end 106. The system 100 may include a first expandable portion 108 that is affixed to the second end 106 of the elongated tube 102. The first expandable portion 108 may be configured to be placed in a patent's uterine cavity through the patient's cervix. The system 100 may further include a second expandable portion 110 that may be disposed between the first expandable portion 108 and the first end 104 of the elongated tube 102. The second expandable portion 110 may be configured to be placed in the patent's cervix upon the first expandable portion 108 being pulled against the proximal aspect of the patient's cervix. Further, the system 100 may include a means for distributing a cervical ripening medication to the patient's cervix. For example, the cervical ripening medication may include dinoprostone, misoprostol, or other medication suitable for introduction to the cervix, such as during labor.

The elongated tube 102, the first expandable portion 108, and the second expandable portion 110 may be manufactured out of silicone or plastic. Additionally, the elongated tube 102, the first expandable portion 108, and the second expandable portion 110 may include other material suitable for intra-bodily exposure, including other surgical grade materials, and the like.

It is contemplated that users of the present disclosure may include obstetricians, gynecologists, physician support staff, and like trained personnel.

The first end 104 of the elongated tube 102 may be configured to have a first port 150, a second port 160, and a third port 170. The first port 150 may be configured to receive a device for filling or inflating the first expandable portion 108 with sterile water, contrast, or other suitable substance sufficient to fill or inflate the first expandable portion 108. A dedicated lumen may connect the first port 150 to the first expandable portion 108. For instance, the substance utilized to fill or inflate the first expandable portion 108 may be an innocuous/non-toxic substance to avoid injury to a patient, such as if the integrity of the material comprising system 100 fails.

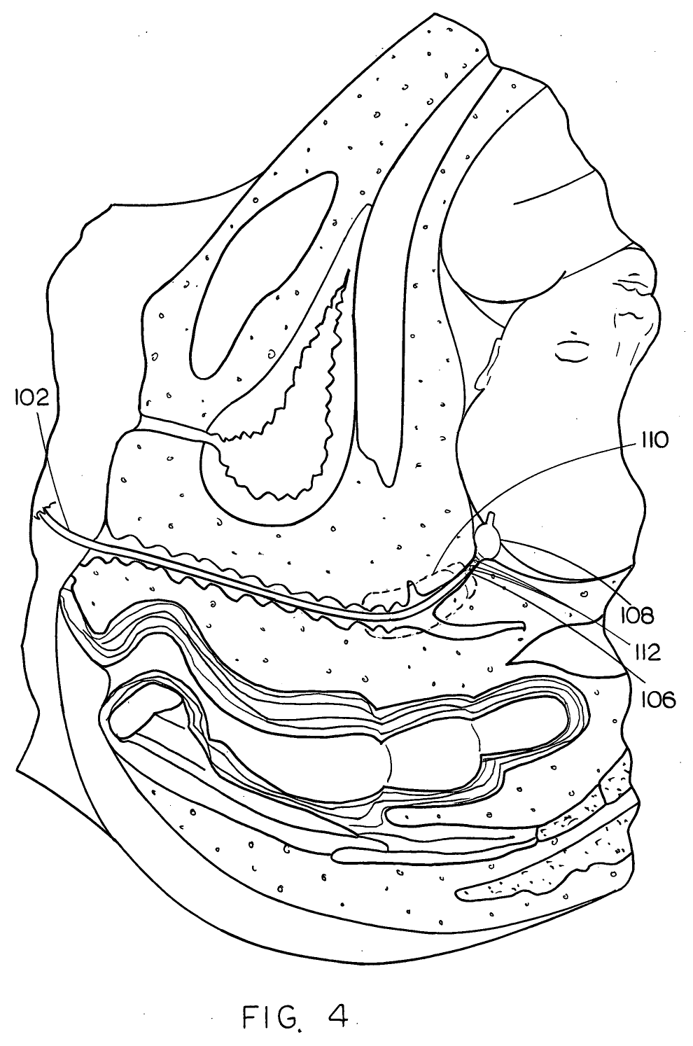

The second port 160 may be configured to receive a device for filling or inflating the second expandable portion 110 with sterile water, contrast, or other suitable substance sufficient to fill or inflate the second expandable portion 110. A dedicated lumen may connect the second port 160 to the second expandable portion 110. For instance, FIG. 4 displays an embodiment of system 100, where the first expandable portion 108 is filled and pulled against the proximal aspect of the cervix, and the second expandable portion 110 is displayed in an unfilled state, and in a filled state, as shown by the dashed outline. In one embodiment, a Leveen like pressure inflating device 180 or the like may be used to control the filling or inflating of the second expandable portion 110 via the second port 160. A user may control the Leveen like pressure inflating device 180 by operating a piston 182 located on the Leveen like pressure inflating device 180. The second expandable portion 110 may increase in pressure, which may inflate the second expandable portion 110 and dilate the patient's cervix. The Leveen like pressure inflating device 180 may be similar to the device described in Marshall L. Stoller et al., Urinary Stone Disease: The Practical Guide to Medical and Surgical Management, 593 (2007), which is incorporated herein by reference.

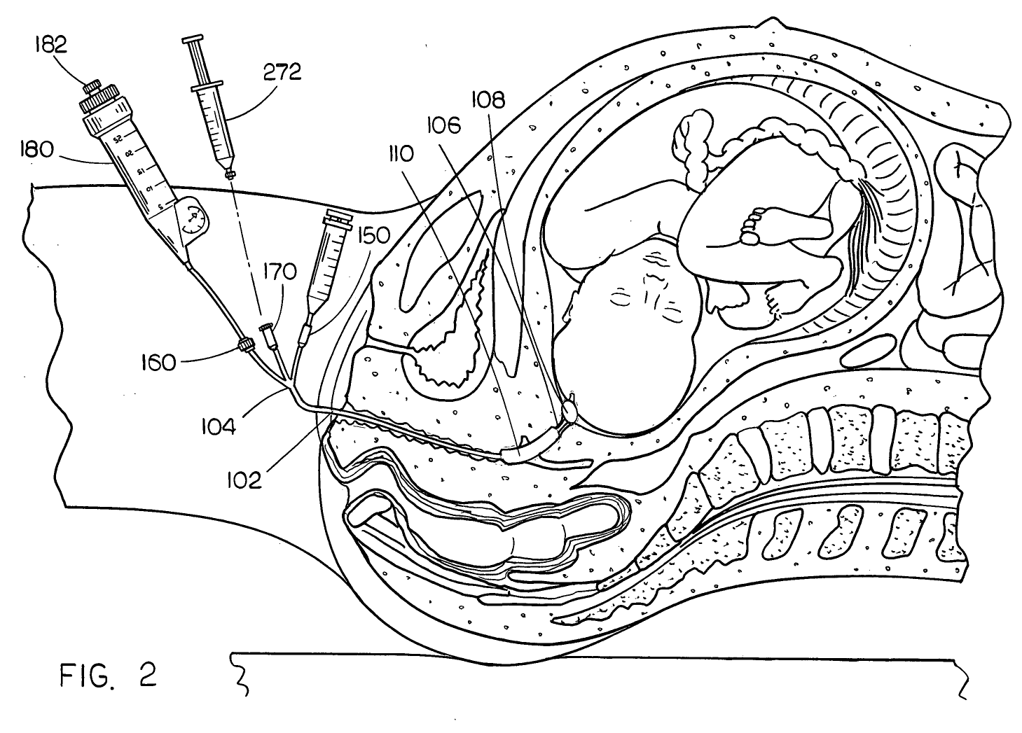

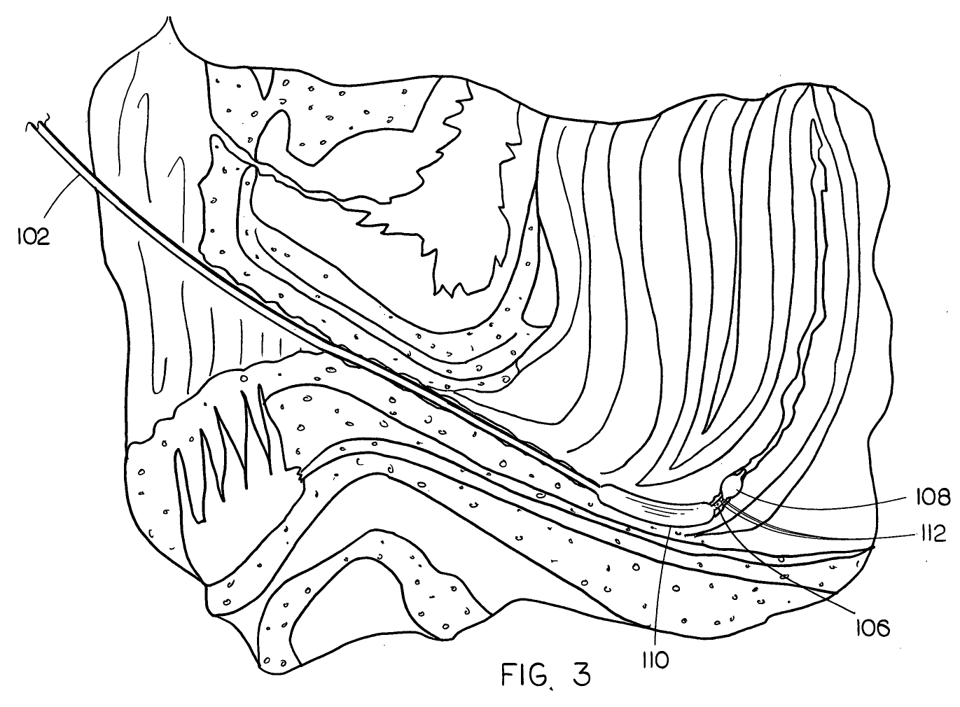

The third port 170 may be configured to give a user access to a lumen. The user may utilize a syringe or the like to insert into the third port 170 and disperse a cervical ripening medication into a dedicated lumen or channel in the elongated tube 102. A plurality of holes 112 or openings may be located between the first expandable portion 108 and the second expandable portion 110 on the elongated tube 102. Holes 112 may allow the dispersal of the cervical ripening medication into the cervix upon dispensation of the cervical ripening medication, such as via a syringe 272 (FIG. 2) or the like. This dispersal may occur after the first expandable portion 108 has been placed in the patient's uterine cavity, inflated, and pulled against the distal end of the cervix (as shown in FIGS. 2 and 3) and after the second elongated tube 110 has been inflated, dilating the cervix. For instance, when the first expandable portion 108 is pulled against the distal end of the cervix, the uterine cavity may be effectively sealed from the cervix, thereby preventing the cervical ripening medication from entering the uterine cavity.

The first expandable portion 108 may be a generally spherical shaped silicone balloon, such as when inflated. The first expandable portion 108 may be inflated to a volume that does not allow the first expandable portion 108 to be pulled through the cervix upon the user pulling the first expandable portion 108 against the distal end of the cervix, as shown in FIGS. 2 and 4. For instance, in one embodiment, the diameter of the first expandable portion 108, when inflated, may be larger than the diameter of the cervix. It may be appreciated that the first expandable portion 108 may include a semi-rigid material, such that the diameter of the first expandable portion 108 may vary depending on the pressure exerted by the substance utilized to fill or inflate. Thus, the first expandable portion 108 may be utilized in many stages of labor, where the diameter of the cervix may change over time.

The second expandable portion 110 may be a generally cylindrical shaped silicone balloon, such as when inflated. The second expandable portion 110 may be properly positioned in the cervix upon the first expandable portion 108 being placed in the uterine cavity and pulled against the inner cervix. An alternative means for delivering the cervical ripening medication may include coating the second expanding portion 110 with the cervical ripening medication. For example, coating may include chemical bonding, adhering, dipping, spray coating, and the like. Thus, when the second expandable portion 110 is filled or inflated to exert pressure on the cervical walls, the cervical ripening medication may directly contact the cervical walls. Another alternative means for delivering the cervical ripening medication includes positioning a disc coated with or including cervical ripening medication at the proximal side of the system 100, such as near the first end 104. When the first expandable portion 108 and the second expandable portion 110 are expanded in the uterine cavity and the cervix, respectively, the disc may be guided along the elongated tube 102 and placed into the cervix.

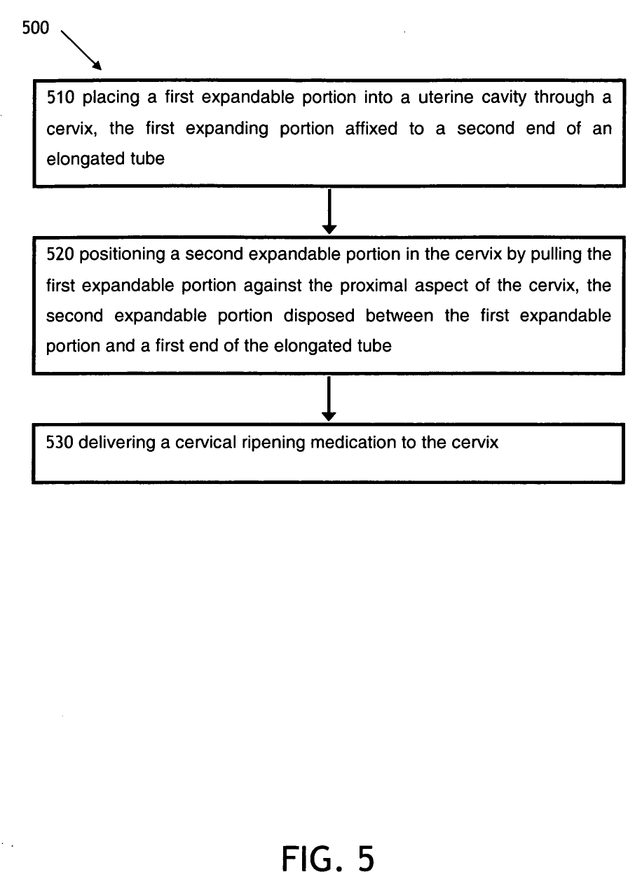

Referring to FIG. 5, a diagram illustrating a method 500 for delivering a cervical ripening medication is illustrated. Method 500 may include placing a first expandable portion into a uterine cavity through a cervix, the first expanding portion affixed to a second end of an elongated tube 510. Method 500 may also include positioning a second expandable portion in the cervix by pulling the first expandable portion against the proximal aspect of the cervix, the second expandable portion disposed between the first expandable portion and a first end of the elongated tube 520. Method 500 may further include delivering a cervical ripening medication to the cervix 530.

Alternatively, step 510 may include placing a balloon of a generally spherical shape when inflated into a uterine cavity through a cervix. Step 510 may also include placing a first expandable portion manufactured of silicone into a uterine cavity through a cervix, the first expanding portion affixed to a second end of an elongated tube.

Step 520 may alternatively include positioning a balloon of a generally cylindrical shape when inflated in the cervix by pulling the first expandable portion against the proximal aspect of the cervix, the balloon of a generally cylindrical shape when inflated disposed between the first expandable portion and a first end of the elongated tube. Step 520 may also include positioning a second expandable portion manufactured of silicone in the cervix by pulling the first expandable portion against the proximal aspect of the cervix, the second expandable portion manufactured of silicone disposed between the first expandable portion and a first end of the elongated tube.

Step 530 may alternatively include delivering a cervical ripening medication to the cervix, wherein the second expandable portion is coated with the cervical ripening medication. Step 530 may also include delivering a cervical ripening medication to the cervix via a disc coated with the cervical ripening medication placed in the cervix, the disc being guided by the elongated tube.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

What is claimed is:

1. A cervical dilating and ripening catheter system, comprising:
an elongated tube having a first end and a second end;
a first expandable portion affixed to the second end of the elongated tube, the first expandable portion configured to be placed in a uterine cavity through a cervix;
a second expandable portion disposed between the first expandable portion and the first end of the elongated tube, the second expandable portion configured to be placed in the cervix upon the first expandable portion being pulled against the proximal aspect of the cervix; and
means for distributing a cervical ripening medication to the cervix.

2. The cervical dilating and ripening catheter system of claim 1, wherein the first expandable portion is a balloon of a generally spherical shape when inflated.

3. The cervical dilating and ripening catheter system of claim 1, wherein at least one of the first expandable portion or second expandable portion is manufactured of silicone.

4. The cervical dilating and ripening catheter system of claim 1, wherein the second expandable portion is a balloon of a generally cylindrical shape when inflated.

5. The cervical dilating and ripening catheter system of claim 1, wherein the means for distributing a cervical ripening medication to the cervix is accomplished by coating the second expandable portion with the cervical ripening medication.

6. A method for delivering a cervical ripening medication, comprising:
placing a first expandable portion into a uterine cavity through a cervix, the first expanding portion affixed to a second end of an elongated tube;
positioning a second expandable portion in the cervix by pulling the first expandable portion against the proximal aspect of the cervix, the second expandable portion disposed between the first expandable portion and a first end of the elongated tube; and
delivering a cervical ripening medication to the cervix.

7. The method for delivering a cervical ripening medication of claim 6, wherein delivering a cervical ripening medication to the cervix includes:
delivering a cervical ripening medication to the cervix, wherein the second expandable portion is coated with the cervical ripening medication.

8. The method for delivering a cervical ripening medication of claim 6, wherein delivering a cervical ripening medication to the cervix includes:
delivering a cervical ripening medication to the cervix via a disc coated with the cervical ripening medication placed in the cervix, the disc being guided by the elongated tube.

9. The method for delivering a cervical ripening medication of claim 6, wherein placing a first expandable portion into a uterine cavity through a cervix includes:
placing a balloon of a generally spherical shape when inflated into a uterine cavity through a cervix.

10. The method for delivering a cervical ripening medication of Claim 6, wherein positioning a second expandable portion in the cervix by pulling the first expandable portion against the proximal aspect of the cervix, the second expandable portion disposed between the first expandable portion and a first end of the elongated tube includes:
positioning a balloon of a generally cylindrical shape when inflated in the cervix by pulling the first expandable portion against the proximal aspect of the cervix, the balloon of a generally cylindrical shape when inflated disposed between the first expandable portion and a first end of the elongated tube.

11. The method for delivering a cervical ripening medication of claim 6, wherein placing a first expandable portion into a uterine cavity through a cervix, the first expanding portion affixed to a second end of an elongated tube includes:
placing a first expandable portion manufactured of silicone into a uterine cavity through a cervix, the first expanding portion affixed to a second end of an elongated tube.

12. The method for delivering a cervical ripening medication of claim 6, wherein positioning a second expandable portion in the cervix by pulling the first expandable portion against the proximal aspect of the cervix, the second expandable portion disposed between the first expandable portion and a first end of the elongated tube includes:
positioning a second expandable portion manufactured of silicone in the cervix by pulling the first expandable portion against the proximal aspect of the cervix, the second expandable portion manufactured of silicone disposed between the first expandable portion and a first end of the elongated tube.

13. A cervical dilating and ripening catheter system, comprising:
an elongated tube having a first end and a second end;
a first balloon affixed to the second end of the elongated tube, the first balloon configured to be placed in a uterine cavity through a cervix;
a second balloon disposed between the first balloon and the first end of the elongated tube, the second balloon configured to be placed in the cervix upon the first balloon being pulled against the proximal aspect of the cervix; and
a lumen for distributing a cervical ripening medication to the cervix.

14. The cervical dilating and ripening catheter system of claim 13, wherein the first balloon is a generally spherical shape when inflated.

15. The cervical dilating and ripening catheter system of claim 13, wherein the second balloon is a generally cylindrical shape when inflated.

16. The cervical dilating and ripening catheter system of claim 13, further including:
a first port configured to receive a device for filling or inflating the first balloon.

17. The cervical dilating and ripening catheter system of claim 13, wherein the first balloon is inflated with at least one of sterile water or contrast.

18. The cervical dilating and ripening catheter system of claim 13, further including:
a second port configured to receive a device for filling or inflating the second balloon.

19. The cervical dilating and ripening catheter system of claim 13, wherein the second balloon is inflated with at least one of sterile water or contrast.

20. The cervical dilating and ripening catheter system of claim 13, wherein the lumen distributes the cervical ripening medication to the cervix through a plurality of holes disposed between the first balloon and the second balloon.
